(19) United States
(12) Patent Application Publication
(10) Pub. No.: US 2026/0294279 A1
(43) Pub. Date: Oct. 1, 2026

(54) METHODS AND SYSTEMS FOR USING REAL WORLD OBJECTS FOR FUNCTIONAL THERAPY IN AN EXTENDED REALITY ENVIRONMENT

(71) Applicant: Neurovirt Limited, London (GB)

(72) Inventors: Kevin Kuhn Agnes, Porto Alegre (BR); Gregoriou Evridiki, London (GB)

(21) Appl. No.: 19/476,965

(22) PCT Filed: Mar. 8, 2024
(86) PCT No.: PCT/GB2024/050618

§ 371 Date: Oct. 20, 2025

(30) Foreign Application Priority Data
Apr. 19, 2023 (GB) 2305754.0

(51) Int. Cl. A61B 5/11; A61B 90/00

(52) U.S. Cl. CPC A61B 5/1125; A61B 5/1128; A61B 90/36; A61B 2090/365

(57) ABSTRACT

The claimed invention provides methods and systems for using real world objects for functional therapy in an extended reality environment, the method comprising: identifying a fully extended position of one or more digits of a user's hand when the user is holding a real-world object; identifying a second position of the one or more digits of the user's hand when the user is determined to have gripped the real-world object as tightly as possible; determining a third position of the one or more digits of the user's hand between the fully extended position and the second position and setting a threshold based on the third position; and upon determining that the position of the one or more digits of the user's hand have moved from the first position past the threshold, representing a first action within the extended reality environment

### FIELD

Aspects and embodiments of the disclosure relate to methods and systems for using real world objects for functional therapy in an extended reality environment.

### BACKGROUND

The advancement of technology provides opportunity for bringing technology solutions to the forefront of healthcare challenges. One such example is the rehabilitation of patients suffering from neurological conditions. For example, patients having suffered a stroke may experience reduced motor control or movement impairment. The manifested symptoms can have a significant impact on the patient's quality of life and ability to undertake certain tasks.

It has long been recognized that haptic feedback is beneficial in helping rehabilitate patients to recover motor control and reduce the impact of movement on their everyday lives. Haptic feedback, which refers to the use of touch or tactile sensations to communicate with humans or machines, has been used in various therapeutic settings to improve outcomes for patients. In the case of occupational therapy, haptic feedback can be used to help patients who have difficulty with fine motor skills or sensory processing issues. For example, therapists may use haptic feedback devices to provide patients with sensory input to help them better understand how to hold and manipulate objects. In the case of physical therapy, haptic feedback can be used in physical therapy to provide patients with a better sense of balance and body position. For example, balance boards with haptic feedback can help patients improve their posture and balance.

Use of haptic feedback in an extended reality environment for healthcare is not new. Applications exist to help provide physical sensation to increase the user's sense of presence and to make movement rehabilitation more effective and engaging. Haptic feedback can also provide real-time feedback on the user's movements. This has been recognized as beneficial in helping patients correct movement impairment and to improve motor function.

Haptic feedback in extended reality environments is generally limited to the provision of vibratory feedback through a headset or controllers. Extended reality can provide highly realistic experiences, but effective therapy requires interaction with everyday physical objects such as inserting a key into a lock and practicing the motion of locking and unlocking the lock. While such movement can be simulated in extended reality, it is not the same as feeling the sensation of the key entering the lock and the slight resistance provided by the lock tumblers. While it would be possible to develop specific hardware to provide a realistic haptic experience, the cost of doing so for many everyday tasks is prohibitive.

Hardware and methods exist to integrate objects into an extended reality environment. Examples include: i) development of dedicated objects that include internal sensors and are directly coded into an extended reality application; ii) add-ons for extended reality controllers, for example a gun in which the controller is places; and iii) attachment of sensors, i.e., IMUs, to existing objects. None of these existing methods (and associated hardware) is usable for the purpose of strength training within an extended reality environment.

The present invention uses hand tracking technology in an extended reality environment. Such technology is already used in connection with extended reality applications for the purpose of gesture control and interaction with virtual objects (see US2021264140). Some existing applications determine the position of a user's hand in a real-world space for the purpose of interacting with an object i.e., piano keys as disclosed in US20220375362. However, the known prior art is not suitable for use in movement rehabilitation using extended reality hardware and software.

It is against this background that the present invention has arisen.

### SUMMARY

An aspect of the present disclosure provides, a method for using real world objects for functional therapy in an extended reality environment, the method comprising: identifying a fully extended position of one or more digits of a user's hand when the user is holding a real-world object; identifying a second position of the one or more digits of the user's hand when the user is determined to have gripped the real-world object as tightly as possible; determining a third position of the one or more digits of the user's hand between the fully extended position and the second position and setting a threshold based on the third position; and upon determining that the position of the one or more digits of the user's hand have moved from the first position past the threshold, representing a first action within the extended reality environment.

The claimed uses a real-world object, i.e., a stress ball, for functional therapy. This approach has several advantages, including familiarity with the object, ease of use, and low cost. By using a real-world object, a more engaging and immersive therapy experience is provided to the user. This can lead to improved therapy outcomes, as the user is more likely to stay engaged and motivated during the therapy session, particularly where the therapy session is delivered via an extended reality environment. Furthermore, the claimed method sets a threshold based on the user's individual finger mobility and grip, which can help to personalize the therapy experience and ensure that the user is appropriately challenged during the therapy session. This can help the therapist to better understand the user's progress and adjust the therapy plan as needed through provision of stress balls having different densities or sizes, for example. One embodiment of the claimed invention aids users to increase their grip strength through integration of a stress ball into an extended reality environment. The stress ball provides resistance and consequently helps the user to train their grip strength. As a user progresses through their treatment program, the therapist or user may elect to use stress balls having differing properties in order to increase (or decrease) the level of resistance or difficulty squeezing the stress ball. Similarly, the threshold may also be adjusted in accordance with the user's progress and translated into the extended reality environment.

Implementations may include one or more of the following features.

The method may further include determining that the position of the one or more digits of the user's hand has moved from the second position past the threshold and representing a second action within the extended reality environment.

The position of the one or more digits of the user's hand may be monitored using optical hand tracking techniques.

The one or more digits of the user's hand may include at least the index finger and the thumbs.

The plurality of measurements of the fully extended position and second position may be measurements of MCP, PIP, and DIP angles of one or more digits of the user's hand.

The third position may be 30-80% between the fully extended position and the second position. Preferably, the third position may be 70% between the fully extended position and the second position.

The optical hand tracking technique may include tracking the average angle of the index finger and thumb of the user's hand. Furthermore, the average angle of one or more of the proximal interphalangeal joint (PIP joint), the distal interphalangeal joint (DIP joint), and the metacarpophalangeal joint (MCP joint) is tracked.

The method may include defining a tracking area within the extended reality environment and providing instructions to the user to move their hand into the tracking area, where upon removal of the user's hand from the tracking area, a prompt is generated for display to the user within the extended reality environment to direct the user to return their hand into the tracking area.

The method may include determining the rotational position of the user's hand and providing directions to the user to move their hand into a target rotational position, where upon rotation of the hand into a different position, a prompt is generated for display to the user within the extended reality environment to direct the user to return their hand to the target rotational position.

The average of the fully extended position, second position, and third position may be stored in the memory of an extended reality device, computing device, or cloud storage.

The real-world object may be a stress ball.

The first action and/or second action may be represented in the extended reality environment graphically, audibly, or via haptic feedback.

The method may include assigning a confidence value to the average angle of the joints of each digit of the user's hand, where the confidence value is assigned to trusted if the determined angle of the joints of a digit is trusted, and not trusted if the determined angle of the joints of a digit is not trusted, and where the steps of identifying the fully extended and/or second positions of each digit of the user's hand are repeated for each digit that is assigned a confidence value of not trusted.

The method may include assigning a confidence value to the average angle of the joints of each digit of the user's hand, wherein the confidence value is assigned to trusted if the determined angle of the joints of a digit is trusted, and not trusted if the determined angle of the joints of a digit is not trusted, and wherein a single average value is determined based on the measured angles of digits of the user's hand that are assigned a trusted confidence value.

Another aspect of the invention provides a method of tracking finger position in an extended reality, the method comprising: identifying a fully extended position of one or more digits of a user's hand; identifying a gripped position of the one or more digits of the user's hand; determining a third position of the one or more digits of the user's hand between the fully extended position and the gripped position and setting a threshold based on the third position; and upon determining that the position of the one or more digits of the user's hand have moved from the first position past the threshold, representing a first action within the extended reality environment.

Other embodiments of this aspect include corresponding computer systems, apparatus, and computer programs recorded on one or more computer storage devices, each configured to perform the actions of the methods.

### BRIEF DESCRIPTION OF THE DRAWINGS

The above and other objects and advantages of the invention of the disclosures will be apparent upon consideration of the following detailed description, taken in conjunction with the accompanying drawings in which:

FIGS. 1a and 1b illustrate an implementation of the claimed invention.

FIG. 2 is a flowchart of a method of calibrating a device according to the disclosure.

FIG. 3 is a flowchart of a method of operation during engagement with an extended reality game.

FIG. 4 is a flowchart of a method of using real world objects in an extended reality environment.

### DETAILED DESCRIPTION

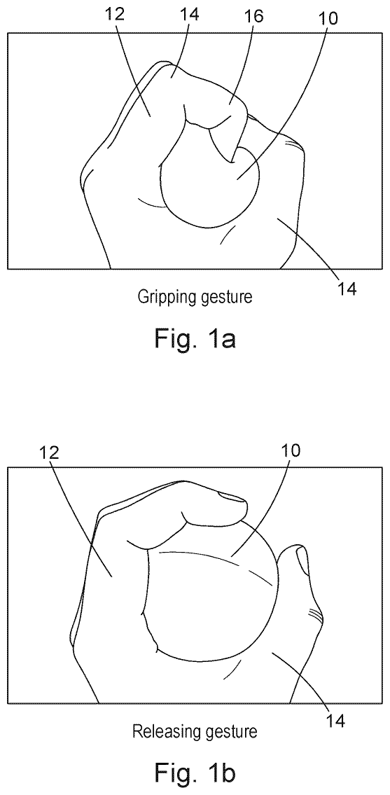

FIGS. 1a and 1b illustrate a particular use case of the claimed invention. One implementation is the incorporation of a stress ball 10 into functional therapy delivered through an extended reality environment. Through squeezing of a stress ball 10, a user's grip strength can be trained. The present invention uses optical tracking techniques to determine the angle of a user's finger joints 12 around the stress ball 10, or another object. Using the angular position of the user's finger joints, the claimed method determines whether the user's fingers 12 are fully extended or in a gripping position. Through taking the average angular orientation of the user's finger joints across multiple measurements, a position between the fully extended and gripped position of the user's fingers 12 can be determined for a specific object. Consequently, when the user grips the stress ball 10 and the angle of his/her finger joints pass the determined position between the fully extended and gripped position, the user's interaction with the stress ball 10 is translated into an extended reality environment. For example, the action of gripping and releasing the stress ball 10 may be interpreted as squeezing and releasing the trigger of a gun to fire at a target.

Through holding the stress ball 10 in a particular way, the user's entire index finger 12 and thumb 14 remains visible as the stress ball 10 is squeezed and released. The three finger joints on a human finger are called the proximal interphalangeal joint (PIP joint), the distal interphalangeal joint (DIP joint), and the metacarpophalangeal joint (MCP joint). The MCP joint is located at the base of the finger, where it meets the hand. The PIP joint is in the middle of the finger, and the DIP joint is at the tip of the finger. The human thumb has just a MCP joint and a DIP joint. As can be seen in FIGS. 1a and 1b, the angle of the PIP joint 14 and DIP joint 16 of the index finger become more acute as the stress ball 12 is squeezed and more obtuse as the stress ball is released.

The angular orientation of the joints of the user's index finger 12 and thumb 14 is determined using an optical camera which may be integral to an extended reality headset. To ensure that the determined angular orientation is accurate to a high degree of accuracy, the user is prompted to move their hand to a particular position within the field of view of the camera. To achieve this, a box may be generated for display within the extended reality environment. If the user's hand is not placed within the box, the user may be prompted to move their hand so that it is placed within the box. In some embodiments, the angular orientation of the user's hand may be determined by the optical camera to ensure that the index finger and thumb of the user's hand are visible to the optical camera. In the event that the angular orientation of the user's hand is determined to be sub-optimal, a prompt may be displayed to the user to instruct them to rotate their hand to an optimal angle.

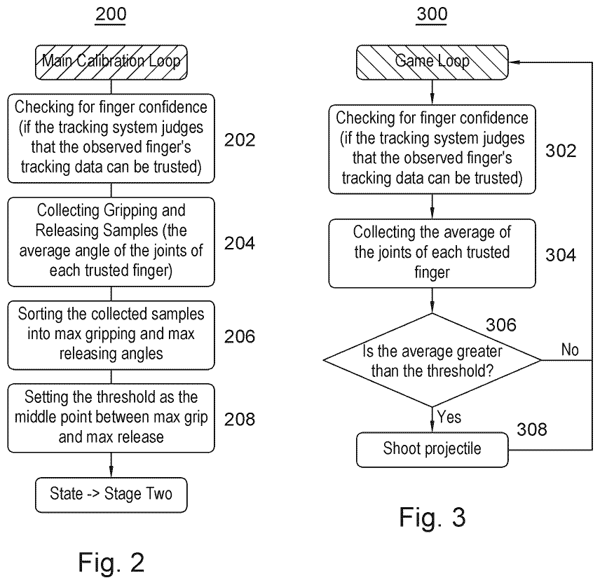

As shown in the flow chart of FIG. 2, an extended reality device is calibrated prior to allowing the user to play a game. This is necessary to ensure that each time a user squeezes the stress ball 10, this motion is identified so that the act of squeezing the stress ball 10 can be translated into the game. Starting at step 202, the device determines whether there is confidence in the tracking data for a user's finger 12 and/or thumb 14. At step 204 the device prompts the user to repeatedly grip and release the stress ball 10. At step 206 the average angle of at least one of the MCP joint, PIP joint, and DIP joint of the user's index finger 12 is determined at each of a fully extended and grip position. At step 208 a position between the average angles of the MCP joint, PIP joint, and DIP joint at the full extended and grip positions of the user's index finger is determined. A threshold is set based on the determined position.

One method of determining the confidence in the tracking data comprises first determining whether the extended reality device is capable of accurate finger tracking. If it is the angular position of each finger is compared to the calibration data. If the measured value falls within the range calculated during the calibration process, the measured value is considered to be trusted.

As shown in the flow chart of FIG. 3, during game play, the device at step 302 tracks the angular position of the MCP joint, PIP joint, and DIP joint of the user's index finger 12 and determines whether the angular position of the user's index finger can be trusted. At step 304 the device determines the average angular position of the user's index finger. At step 306, the average angular position of the user's index finger is compared to the threshold. At step 308, if the angular position of the user's index finger is greater than the threshold, i.e., if it is determined that the user has squeezed the stress ball, the game is instructed to take a specified action, i.e., firing a gun.

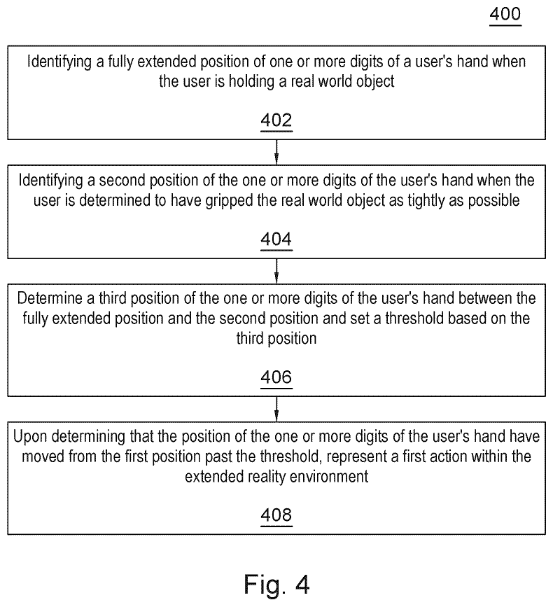

As shown in FIG. 4, a method according to the present disclosure is illustrated. At step 402 the device identifies a fully extended position of one or more digits of a user's hand when the user is holding a real-world object. At step 404 the device identifies a second position of the one or more digits of the user's hand when the user is determined to have gripped the real-world object as tightly as possible. At step 406 determining a third position of the one or more digits of the user's hand between the fully extended position and the second position and setting a threshold based on the third position. At step 408 the device, upon determining that the position of the one or more digits of the user's hand have moved from the first position past the threshold, represents a first action within the extended reality environment. The threshold may be set at a fixed percentage, i.e., 70%, of the angular distance between the fully extended position and the gripped position. Alternatively, the threshold may be dynamically adjusted for each user based on capability and/or rehabilitation regimes. For example, a patient with limited ability to extend and grip their fingers may not be capable of squeezing a stress ball tightly enough to move their fingers past the threshold. In this scenario, the threshold may be adjusted in accordance with the user's capabilities. In another scenario, a user may initially suffer from difficulties extending and gripping their fingers but over time such difficulties may become less pronounced. In this situation it is desirable to adjust the threshold to make the user work harder.

The foregoing disclosure provides illustration and description but is not intended to be exhaustive or to limit the implementations to the precise form disclosed. Modifications may be made considering the above disclosure or may be acquired from practice of the implementations. As used herein, the term “component” is intended to be broadly construed as hardware, firmware, or a combination of hardware and software. It will be apparent that systems and/or methods described herein may be implemented in different forms of hardware, firmware, and/or a combination of hardware and software. The actual specialized control hardware or software code used to implement these systems and/or methods is not limiting of the implementations. Thus, the operation and behavior of the systems and/or methods are described herein without reference to specific software code—it being understood that software and hardware can be used to implement the systems and/or methods based on the description herein. As used herein, satisfying a threshold may, depending on the context, refer to a value being greater than the threshold, greater than or equal to the threshold, less than the threshold, less than or equal to the threshold, equal to the threshold, and/or the like, depending on the context. Although combinations of features are recited in the claims and/or disclosed in the specification, these combinations are not intended to limit the disclosure of various implementations. In fact, many of these features may be combined in ways not specifically recited in the claims and/or disclosed in the specification.

Although each dependent claim listed below may directly depend on only one claim, the disclosure of various implementations includes each dependent claim in combination with every other claim in the claim set. No element, act, or instruction used herein should be construed as critical or essential unless explicitly described as such. Also, as used herein, the articles “a” and “an” are intended to include one or more items and may be used interchangeably with “one or more.” Further, as used herein, the article “the” is intended to include one or more items referenced in connection with the article “the” and may be used interchangeably with “the one or more.” Furthermore, as used herein, the term “set” is intended to include one or more items (e.g., related items, unrelated items, a combination of related and unrelated items, and/or the like), and may be used interchangeably with “one or more.” Where only one item is intended, the phrase “only one” or similar language is used. Also, as used herein, the terms “has,” “have,” “having,” or the like are intended to be open-ended terms. Further, the phrase “based on” is intended to mean “based, at least in part, on” unless explicitly stated otherwise. Also, as used herein, the term “or” is intended to be inclusive when used in a series and may be used interchangeably with “and/or,” unless explicitly stated otherwise (e.g., if used in combination with “either” or “only one of”).

1. A method for using real-world objects for functional therapy in an extended reality environment, the method comprising:
identifying a fully extended position of one or more digits of a user's hand when the user is holding a real-world object;
identifying a second position of the one or more digits of the user's hand when the user is determined to have gripped the real-world object as tightly as possible;
determining a third position of the one or more digits of the user's hand between the fully extended position and the second position and setting a threshold based on the third position; and
upon determining that the position of the one or more digits of the user's hand have moved from the first position past the threshold, representing a first action within the extended reality environment.

2. The method of claim 1 further comprising the collection of a plurality of measurements of the fully extended position and second position and calculating an average of each of the fully extended position and second position, wherein the threshold is set based on the third position being defined between the average of the fully extended position and the average of the second position.

3. The method of claim 2, wherein the plurality of measurements of the fully extended position and second position are measurements of MCP, PIP, and DIP angles of one or more digits of the user's hand.

4. The method of claim 3, wherein the third position is 30-80% between the fully extended position and the second position.

5. The method of claim 1 further comprising determining that the position of the one or more digits of the user's hand have moved from the second position past the threshold and representing a second action within the extended reality environment.

6. The method of claim 1, wherein the position of the one or more digits of the user's hand is monitored using optical hand tracking techniques.

7. The method of claim 5, wherein the one or more digits of the user's hand comprise at least the index finger and the thumbs.

8. The method of claim 6, wherein the optical hand tracking technique comprises tracking the average angle of the index finger and thumb of the user's hand.

9. The method of claim 1 further comprising defining a tracking area within the extended reality environment and providing instructions to the user to move their hand into the tracking area, wherein upon removal of the user's hand from the tracking area, a prompt is generated for display to the user within the extended reality environment to direct the user to return their hand into the tracking area.

10. The method of claim 8 further comprising determining the rotational position of the user's hand and providing directions to the user to move their hand into a target rotational position, wherein upon rotation of the hand into a different position, a prompt is generated for display to the user within the extended reality environment to direct the user to return their hand to the target rotational position.

11. The method of claim 1 further comprising assigning a confidence value to the average angle of the joints of each digit of the user's hand, wherein the confidence value is assigned to trusted if the determined angle of the joints of a digit is trusted, and not trusted if the determined angle of the joints of a digit is not trusted, and wherein the steps of identifying the fully extended and/or second positions of each digit of the user's hand are repeated for each digit that is assigned a confidence value of not trusted.

12. The method of claim 1 further comprising assigning a confidence value to the average angle of the joints of each digit of the user's hand, wherein the confidence value is assigned to trusted if the determined angle of the joints of a digit is trusted, and not trusted if the determined angle of the joints of a digit is not trusted, and wherein a single average value is determined based on the measured angles of digit's of the user's hand that are assigned a trusted confidence value.

13. The method of claim 1, wherein the fully extended position, second position, and third position are stored in the memory of an extended reality device, computing device, or cloud storage.

14. The method of claim 1, wherein the real-world object is a stress ball.

15. The method of claim 1, wherein the first action and/or second action is represented in the extended reality environment graphically, audibly, or via haptic feedback.

16. A method of tracking finger position in an extended reality environment, the method comprising:
identifying a fully extended position of one or more digits of a user's hand;
identifying a gripped position of the one or more digits of the user's hand;
determining a third position of the one or more digits of the user's hand between the fully extended position and the gripped position and setting a threshold based on the third position; and
upon determining that the position of the one or more digits of the user's hand have moved from the first position past the threshold, representing a first action within the extended reality environment.
